(19) United States
(12) Patent Application Publication
(10) Pub. No.: US 2011/0307051 A1
(43) Pub. Date: Dec. 15, 2011

(54) Bioerodible Endoprostheses Including Electrochemical Cell

(75) Inventors: Liliana Atanasoska, Edina, MN (US); Michael J. Root, Lino Lakes, MN (US)

(73) Assignee: BOSTON SCIENTIFIC SCIMED, INC., Maple Grove, MN (US)

(21) Appl. No.: 13/156,940

(22) Filed: Jun. 9, 2011

Related U.S. Application Data
(60) Provisional application No. 61/353,335, filed on Jun. 10, 2010.

(51) Int. Cl. A61F 2/82

(52) U.S. Cl. 623/1.16; 623/1.38

(57) ABSTRACT

A bioerodible endoprosthesis includes a bioerodible body and a bioerodible electrochemical cell. The bioerodible body includes a bioerodible metal. The bioerodible electrochemical cell includes a cathode, an anode, and an electrolyte between the cathode and the anode. The cathode is adapted to be in electrical contact with at least a first portion of the bioerodible body when the electrochemical cell is activated to accelerate the bioerosion of the first portion of the bioerodible body when the endoprosthesis is implanted within a physiological environment.

### CROSS-REFERENCE TO RELATED APPLICATIONS

This application is a non-provisional of and claims priority to U.S. Provisional Application Ser. No. 61/353,335, filed Jun. 10, 2010, the entire contents of which are incorporated herein by reference.

### TECHNICAL FIELD

This disclosure relates to bioerodible endoprostheses that include one or more electrochemical cells that alter the erosion of the endoprosthesis within a physiological environment.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

It is sometimes desirable for an implanted endoprosthesis to erode over time within the passageway. For example, a fully erodible endoprosthesis does not remain as a permanent object in the body, which may help the passageway recover to its natural condition. Bioerodible endoprostheses can be formed from, e.g., a polymeric material, such as polylactic acid, or from a metallic material, such as magnesium, iron or an alloy thereof.

Bioerodible metals can erode due to corrosion in vivo. The corrosion process, however, can be non-uniform due to localized attacks. In vivo corrosion rates are difficult to predict from in vitro data. Accordingly, it is difficult to design a bioerodible endoprosthesis having the desired structural integrity for a desired period of time

### SUMMARY

A bioerodible endoprosthesis is described that includes a bioerodible body and a bioerodible electrochemical cell. The bioerodible body includes a bioerodible metal. The bioerodible electrochemical cell includes a cathode, an anode, and an electrolyte between the cathode and the anode. The cathode is adapted to be in electrical contact with at least a first portion of the bioerodible body when the electrochemical cell is activated to accelerate the bioerosion of the first portion of the bioerodible body when the endoprosthesis is implanted within a physiological environment.

The bioerodible endoprosthesis described herein can provide a bioerodible stent that has an more predictable bioerosion rate. The bioerosion profile can be tailored for a particular use. For example, the endoprosthesis can have a bioerosion profile that delays significant bioerosion until after the endoprosthesis is endothelialized, followed by rapid bioerosion following endothelialization. The endoprosthesis can also be fully bioerodible, with the constituents stent, including of the electrochemical cell(s), all being benign.

The cathode, in some embodiments, includes an iron(VI) compound. For example, the cathode can include K2FeO₄, K₃Na(FeO₄)₂, BaFeO₄, or a combination thereof. The cathode can also include KMnO₄. In some embodiments, the cathode is free of nickel(II) and cobalt(II). The cathode, in some embodiments, includes iron phosphate or an olivine metal phosphate.

The anode, in some embodiments, includes a metal or a metal boride. For example, the anode can be Mg, Zn, MgB₂, FeB, or a combination thereof.

The electrolyte is between the anode and the cathode. In some embodiments, the electrolyte is a metal hydroxide. For example, the electrolyte can include KOH. In other embodiments, the electrolyte includes a polysaccharide polymer and a salt.

The encapsulated electrochemical cell can also include a shield disposed between the electrolyte and the cathode or between the electrolyte and the anode. The shield, for example, can include magnesia and/or zirconia.

The encapsulated electrochemical cell can be bioerodible. The encapsulated electrochemical cell can be designed such that the components react with each other and/or molecules within the physiological environment to be absorbed by the body.

The bioerodible body can include a first bioerodible portion and a second bioerodible portion. The electrochemical cell can be embedded between the first bioerodible portion and the second bioerodible portion such that the cathode is adapted to be in electrical contact with the first bioerodible portion and the anode is adapted to be in electrical contact with the second bioerodible portion when the electrochemical cell is activated. The endoprosthesis can further include an insulating layer between the first bioerodible portion and the second bioerodible portion. The insulating layer can include a polymer. For example, the polymer for the insulating layer can include polylactic acid, poly(lactic-co-glycolic acid), Poly(propylene-ram-ε-caprolactone carbonate), polycaprolactone, poly-L-lactic-acid, poly(3-hydroxybutyrate), or a combination thereof. In some embodiments, the first bioerodible portion and the second bioerodible portion include different bioerodible metals. In other embodiments, the first bioerodible portion and the second bioerodible portion comprise comprises the same bioerodible metal. For example, the first bioerodible portion can include metallic iron or a bioerodible iron alloy.

The endoprosthesis can be a stent. A stent can include a plurality of struts. In some embodiments, at least one strut of a stent can include at least a portion of the first bioerodible portion, at least a portion of the second bioerodible portion, and at least a portion of the encapsulated electrochemical cell.

### DESCRIPTION OF DRAWINGS

FIG. 1 is an example of a stent.

FIG. 2A depicts an idealized degradation profile for bioerodible stents, both coated and non-coated.

FIG. 2B depicts anodic dissolution rates for iron at different current densities.

FIG. 2C depicts anodic dissolution rates for magnesium at different current densities.

FIG. 3 depicts a cross-section of a stent strut having a bioerodible body and an electrochemical cell.

FIG. 4A depicts a prophetic example of a stent strut cross-section having an electrochemical cell according to particular embodiments.

FIG. 4B is a schematic illustration of the redox electrochemistry involved in a prophetic example of an electrochemical cell including an iron (IV) cathode, a KOH electrolyte, and a magnesium boride anode.

FIG. 5A depicts a prophetic example of a stent strut cross-section having an electrochemical cell according to additional embodiments.

FIG. 5B is a schematic illustration of the electrochemical intercalation involved in a prophetic example of an electrochemical cell including a nano-sized higher surface area lipscombite Fe²⁺Fe₂³⁺(PO₄)₂(OH)₂ iron phosphate cathode, a magnesium metal anode, and a polysaccharide polymer electrolyte including a magnesium chloride salt.

FIGS. 6A and 6B schematically illustrate how a stent strut can be implanted within a body lumen and undergo endotheliazation.

FIG. 7 schematically illustrates an endothelized and partially eroded stent strut cross section.

### DETAILED DESCRIPTION

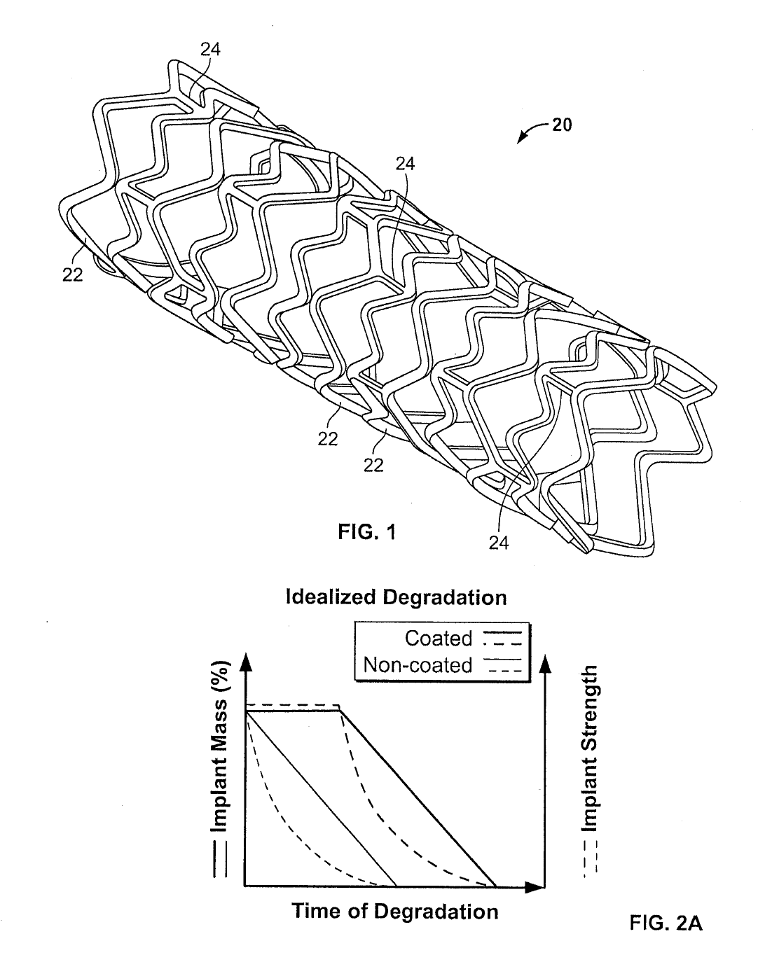

A stent 20, shown in FIG. 1, is discussed below as an example of one endoprosthesis according to the instant disclosure. Stent 20 includes a pattern of interconnected struts forming a structure that contacts a body lumen wall to maintain the patency of the body lumen. For example, stent 20 can have the form of a tubular member defined by a plurality of bands 22 and a plurality of connectors 24 that extend between and connect adjacent bands. During use, bands 22 can be expanded from an initial, small diameter to a larger diameter to contact stent 20 against a wall of a vessel, thereby maintaining the patency of the vessel. Connectors 24 can provide stent 20 with flexibility and conformability that allow the stent to adapt to the contours of the vessel. Other examples of endoprostheses can include covered stents and stent-grafts.

One or more struts of stent 20 is to adapted to erode under physiological conditions. Accordingly, the stent 20 includes a bioerodible body comprising at least one bioerodible metal. Examples of bioerodible metals include magnesium, zinc, iron, and alloys thereof. In some embodiments, the bioerodible body includes iron (e.g., substantially pure iron or iron alloy). Iron alloys can include at least 65% iron. For example, the bioerodible metal portion can include a bioerodible iron alloy that includes up to twenty percent manganese, up to 10 percent silver, and up to five percent carbon.

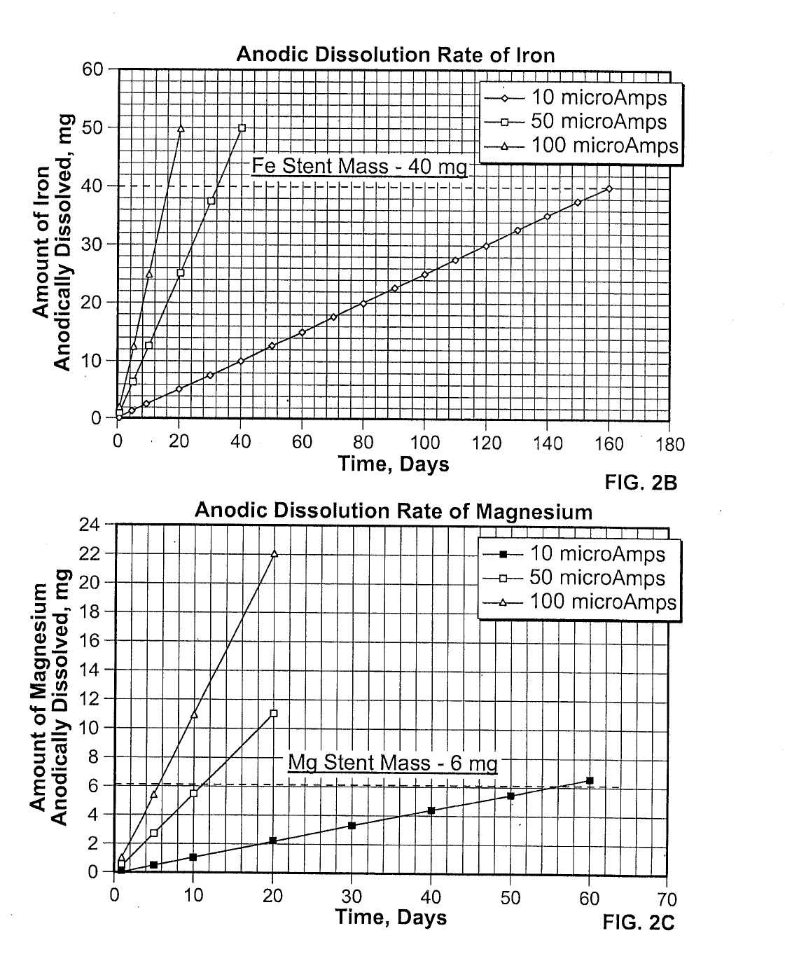

As a stent bioerodes, the stent mass decreases, which also reduces the strength of the stent. As shown in FIG. 2A, while the mass of the stent decreases at an approximately linear rate, the strength of the stent decreases exponentially. As the strength of the stent decreases, portions of the stent may fracture under the loads associated with being implanted within a body lumen (e.g., within a blood vessel). Stent fragments that separate from the stent and that flow within the body lumen can present a hazard (e.g., an embolism). Furthermore, non-uniform corrosion rates can further increase the possibility of larger fragments separating from the stent. Coatings and other techniques can be used to slow or delay the initial degredation of the stent upon implantation within a physiological environment. Delaying the bioerosion processes can allow for the body passage way to heal and for the stent to become endothelialized (surrounded by tissues cells of the lumen wall) before the strength of the stent is reduced to a point where the stent fails under the loads associated with being implanted within a body lumen (e.g., within a blood vessel). When an endothelialized stent fragments, the segments of the stent can be contained by the lumen wall tissue and are thus less likely to be released into the blood stream. Endothelialization can take place as soon as one to two weeks after implantation. Endothelialization can prevent some corrosion, but localized corrosion still occurs, which can result in uneven corrosion. A stent, however, needs to retain its structural integrity even after endothelization to allow for arterial remodeling. In some embodiments, a stent should be capable of maintaining the patency of a blood vessel for about six months before the erosion of the materials of the stent result in the stent becoming compliant with the blood vessel.

Rather than rely on simple galvanic corrosion, including the localized and uneven localized corrosion that occurs after endothelization, the rate of bioerosion of a bioerodible metal can be accelerated and homogenized by applying an electrical current to the bioerodible body by a process known as anodic dissolution. A forced anodic dissolution of a stent by an applied electrochemical battery will enable a metallic material to disappear uniformly by applying positive current. A battery impressed current system can prevent localized corrosion from occurring initially. A battery can also be used to apply a cathodic potential. Cathodic potential can have a healing effect. Cathodic potential can also prevent the formation of a highly alkaline environment where the bioerodible material erodes to form basic byproducts (i.e., where the bioerodible material is magnesium).

The accelerated rate of bioerosion is determined by the current density of the applied to the bioerodible body. For example, FIGS. 2B and 2C depict the anodic dissolution rates for iron and magnesium, respectively. For example, an iron stent having a mass of 40 mg with an applied current density of about 100 μA can completely bioerode in about 5-6 months. For comparison, the same stent implanted without any applied current may still be present 15 months after implantation. Magnesium stents, however, bioerode faster than iron and the application of a current also increases the rate of magnesium bioerosion. As shown in FIG. 2C, an applied current density of about 100 μA to a 6 mg magnesium stent can completely erode that stent in about 6 days, while the same magnesium stent could last over 60 days without the applied current. As shown, the rate of anodic dissolution depends on the applied current density.

Stent 20 includes at least one electrochemical cell 40 that accelerates the bioerosion of at least a portion of the bioerodible body through anodic dissolution. The electrochemical cell 40 can be used to selectively accelerate the bioerosion of the stent. For example, the electrochemical cell 40 can be activated once the stent has become endothelialized. Furthermore, the electrochemical cell 40 can be used to accelerate the bioerosion of select portions of a stent and can result in a more uniform corrosion profile along exterior surfaces of the stent. By an appropriate selection of the various materials for the stent, a stent can be designed with an appropriate bioerosion profile, while ensuring that the bioerosion byproducts are benign.

The electrochemical cell 40 can be bioerodible. Once the bioerodible body has anodically dissolved to directly expose the electrochemical cell 40 to the physiological environment, the different components of the electrochemical cell 40 can bioerode through various other mechanisms. The selection and use of a cathode 42, anode 44, and electrolyte 46 that are all bioerodible when in combination allow for the creation of a fully bioerodible stent 20 having a more uniform and controlled bioerosion profile.

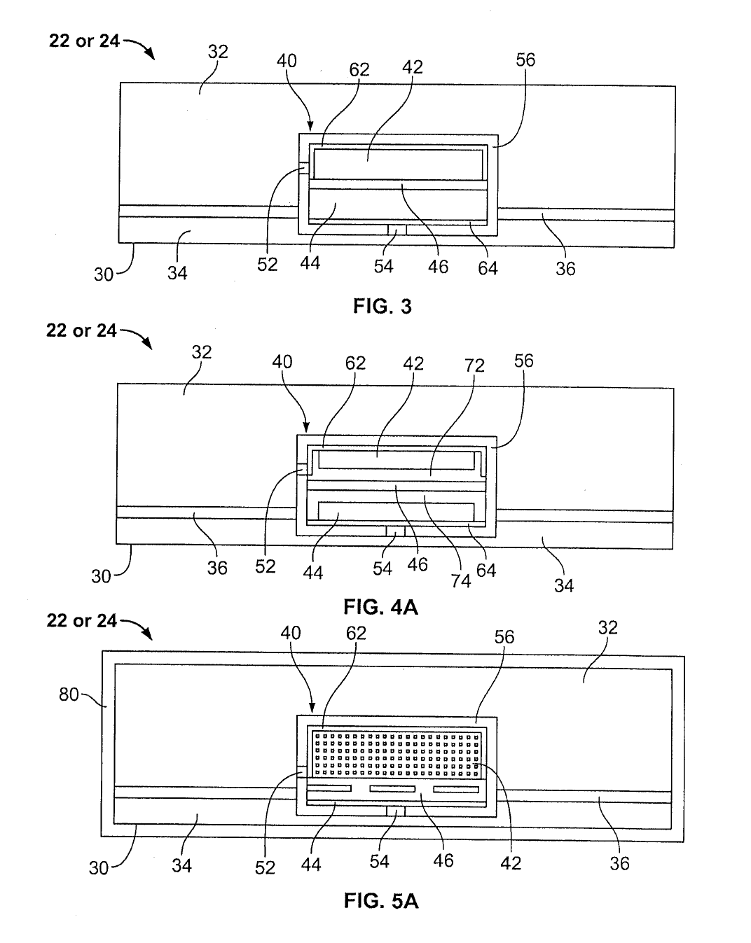

FIG. 3 depicts an example cross-section of a strut (band 22 or connector 24) including a bioerodible body 30 encapsulating an electrochemical cell 40. The electrochemical cell includes a cathode 42, an anode 44, and an electrolyte 46 between the cathode and the anode. The bioerodible body includes first bioerodible metal portion 32 and a second bioerodible metal portion 34. The bioerodible body includes an insulating layer 36 between the first bioerodible metal portion 32 and the second bioerodible metal portion 34 and an encapsulating layer 56 that isolates the electrochemical cell from the bioerodible body 30.

The electrochemical cell 40 is depicted in an off-mode with the encapsulating layer 56 fully encapsulates the electrochemical cell 40 to electrically isolate electrochemical cell 40 from the bioerodible metal of the bioerodible body. When in an off-mode, the electrochemical cell 40 does not accelerate the bioerosion rate of the bioerodible metal. When activated, the electrochemical cell 40 allows for electrical contact between the first bioerodible metal portion 32 and the cathode 42 at cathode contact point 52 and for electrical contact between the second bioerodible metal portion 34 and the anode 44 at anode contact point 54. In some embodiments, contacts 52 and 54 can be preformed into the stent prior to implantation and corrosion can be prevented prior to implantation due to a lack of electrolyte and/or physiological fluid. For example, electrolyte could be added at the time of implant. In other embodiments, contacts 52 and/or 54 can be made after implantation (i.e., one or more days after implantation). For example, contacts 52 and/or 54 could be triggered via a remotely controlled sensor and one or more electromechanical motions. FIG. 3 also depicts optional current collectors that 62 and 64 that facilitate the movement of electrons into and out of the electrochemical cell 40.

The cathode 42 can include an iron (VI) compound. In some embodiments, the iron (VI) compound is an iron (VI) salt or an iron (VI) oxide. Iron (VI) compounds are also known as “super-iron.” Iron (VI) is an unusually high oxidation state of iron and is strongly oxidizing. When an iron (VI) compound is used as a cathode, the iron (VI) is reduced to iron (III), which is more stable. An example electrochemical reaction of an iron (IV) cathode being reduced with a Zn metal anode is shown below:

2MFe^{VI}O₄+3Zn→Fe^{III}₂O₃+ZnO+MZnO₂

Fe^{III}₂O₃ (ferric oxide) is stable and biologically benign. The other reaction products can also be biologically benign. Furthermore, decomposition products of iron (VI) compounds, which can include ferric oxide, are also biologically benign. Accordingly, iron (VI) compounds can be safely used as a cathode material in a fully bioerodible stent. For example, the iron (VI) compound cathode 42 can selected from the group consisting of K₂FeO₄, K₃Na(FeO₄)₂, BaFeO₄, and combinations thereof. In some embodiments, cathode 42 can include KMnO₄ in addition to the iron (VI) compound. KMnO₄ is biologically benign and also facilitates iron (VI) charge transfer by providing additional pathways. In some embodiments, the iron (VI) compound cathode can be free of nickel(II) and/or cobalt(II), which can improve the stability of the iron (VI) compound (e.g., when used with a potassium hydroxide electrolyte).

The cathode 42 can, in other embodiments, include a Fe²⁺Fe₂³⁺(PO₄)₂(OH)₂ iron phosphate, an olivine metal phosphate, or an oxidized form thereof (i.e., FePO₄). For example, the cathode 42 can include nano-sized high surface area lipscombite Fe²⁺Fe₂³⁺(PO₄)₂(OH)₂ iron phosphate. In other examples, the cathode 42 includes a phosphate of an olivine (Mg,Fe)SiO₄.

The anode 44 can be a bioerodible metal. For example, the anode can be magnesium, zinc, or an alloy thereof. In some embodiments, the anode can have the same composition as the first and/or second bioerodible metal portions. In some embodiments, the second bioerodible metal portion 34 can function as the anode. In other embodiments, the anode can be carbon, one or more organic polymers, and/or bismuth.

The anode 44 can, in some embodiments, include a metal boride. For example, the anode 44 can be iron boride or magnesium boride. The reaction product of an anodic boride reaction is sodium borate (Borax), which is approved in some countries (not including the USA) as a food additive.

The electrolyte 46 separates the cathode 42 from the anode 44 and provides free ions. In some embodiments, electrolyte 46 includes a polysaccharide polymer and one or more salts. Suitable metal salts include chlorides such as NaCl, KCl, LiCl, MgCl₂. For example, electrolyte 46 can be pullalan, cellulose, or a combination thereof and magnesium chloride. Polysaccharide polymers are biocompatible and can be made bioerodible. In other embodiments, electrolyte 46 is a metal hydroxide. For example, electrolyte 46 can be potassium hydroxide. Metal hydroxides, such as potassium hydroxide, can also be used to make iron (VI) compounds more stable. Furthermore, relatively small amounts of metal hydroxide electrolyte is required to fully discharge the electrochemical cell 40.

The electrolyte 46 also acts as a separator between the cathode and the anode. In other embodiments, a distinct bioerodible separator layer can be positioned between the cathode and the anode.

The insulating layer 36 and the encapsulating layer 56 are used to electrically isolate selected portions of the stent from each other. The insulating layer 36 and/or the encapsulating layer 56 can include a bioerodible polymer. Examples of suitable bioerodible polymers include be polylactic acid (“PLA”), polyglycolic acid (“PGA”), poly(lactic-co-glycolic acid) (“PLGA”), Poly(propylene-ram-ε-caprolactone carbonate) (“PPCL”), polycaprolactone (“PCL”), poly-L-lactic-acid (“PLLA”), poly(3-hydroxybutyrate) (“PHB”), and combinations thereof. For example, PLA can degrade by hydrolysis to produce lactic acid and PLGA can degrade by hydrolysis to produce lactic acid and glycolic acid. These acidic bioerosion byproducts can help buffer alkaline byproducts from the bioerosion of the bioerodible metal and/or can help neutralize metal hydroxide electrolyte. Other suitable polymers are discussed in U.S. Publication No. 2006/0038027.

The electrochemical cell 40 can also optionally include one or more shields 72 and 74 disposed between the electrolyte and the electrodes. For example, FIG. 3A depicts an example of a stent strut cross-section having an electrochemical cell having a cathode shield 72 disposed between the cathode 42 and the electrolyte 46 and an anode shield 74 disposed between the anode 44 and the electrolyte 46. The shields can allow for the transport of ions, such as hydroxide ions, but prevent direct exposure of the electrode to the electrolyte. For example, metal borides can be unstable in an alkaline media, such as KOH, thus a zirconia or magnesia shield can be used to prevent unwanted reactions between the electrolyte and the anode when using a KOH electrolyte with a metal boride anode.

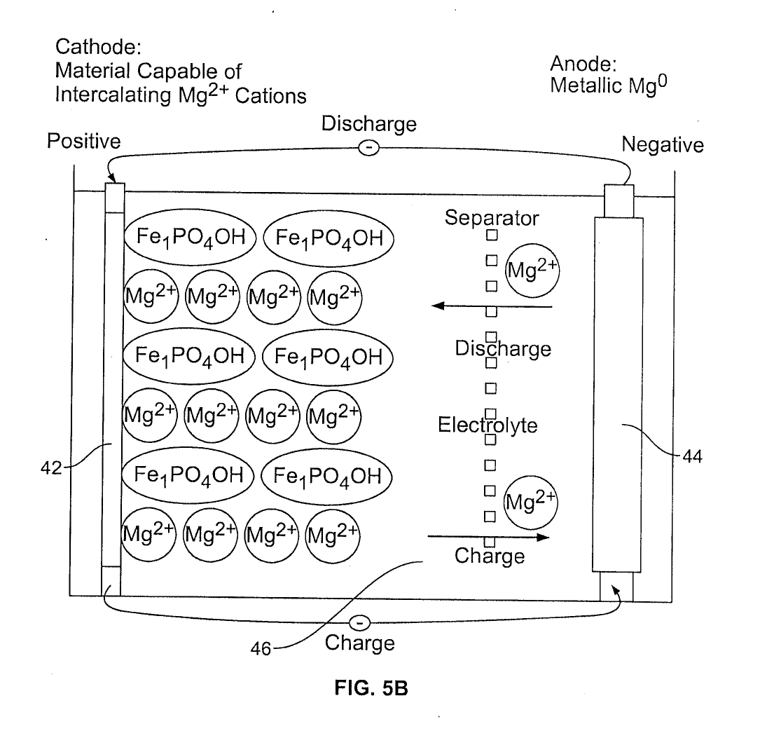

The struts can also include one or more outer layers. An outer layer can also be used to delay bioerosion of the bioerodible body 30. For example, FIG. 5A depicts an example of a stent strut cross-section having an outer layer 80. Outer layer 80 can be composed of a corrosion protecting material, which can be used to delay the bioerosion of at least a portion of the stent 20. In some embodiments, select struts include coating 80. In some embodiments, only certain sides of struts have coating 80. In other embodiments, coating 80 can be on every side of the stent. For example, A biodegradable polymer such as PLA could serve as a corrosion protection layer. In some embodiments, a chelating agents that reacts with magnesium or iron to form one or more water insoluble chelating products is applied to the outer surface of a stent to form a corrosion protection layer. Other suitable materials for a corrosion protecting material include oxides, hydroxides and phosphates of iron, magnesium, and/or calcium. Layer 80 can also include an organic-inorganic hybrid material.

An outer layer can, in some embodiments, include one or more therapeutic agents. For example, a therapeutic agent could be loaded within a polymer matrix and designed to be released from the outer coating over time. In other embodiments, the stent can include one or more therapeutic agents within the bioerodible body 30. The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. In some embodiments, the drug can be incorporated within the porous regions in a polymer coating. Polymers for drug elution coatings are also disclosed in U.S. Published Patent Application No. 2005/019265A. A functional molecule, e.g., an organic, drug, polymer, protein, DNA, and similar material can be incorporated into groves, pits, void spaces, and other features of the stent.

The bioerodible body 30 can include a first bioerodible metal portion 32 and a second bioerodible metal portion 34. Once the electrochemical cell is activated, the cathode 42 is placed in electrical contact with the first bioerodible metal portion 32 and the anode is placed in electrical contact with the second bioerodible metal portion 34 via cathode contact point 52 and anode contact point 54. The electrochemical cell 40 can also include charge collectors 62 and 64 to facilitate transfer of electrons between the electrodes portions of the bioerodible body 30. Because the first bioerodible metal portion 32 will preferentially bioerode while in electrical contact with the cathode 42, the first bioerodible metal portion 32 will eventually erode to remove the electrical connection between the cathode 42and the remainder of the first bioerodible metal portion 32. Accordingly, the placement of the cathode contact point 52 can determine when the anodic dissolution process terminates and the remainder of the stent bioerodes due to other processes. For example, to maximize the anodic dissolution process, one or more cathode contact points 52 can be located along the sides of the encapsulating layer 56.

The first and second bioerodible metal portions are separated by the insulating layer 36 and/or the electrochemical cell 40 (including the encapsulating layer 56) to electrically isolate the first and second bioerodible metal portions. Once activated, the electrochemical cell 40 reacts to provide a driving current that accelerates the corrosion of the first bioerodible metal portion 32 due to its electrical contact with the cathode 42. In other embodiment (not shown), the bioerodible body 30 entirely electrically connected to the cathode and the anode exposed to the physiological environment of the stent once the electrochemical cell 40 is activated. In other embodiments (also not shown), the second bioerodible metal portion 34 can act as the anode, with the electrochemical cell missing an anode internal to an encapsulating layer 56.

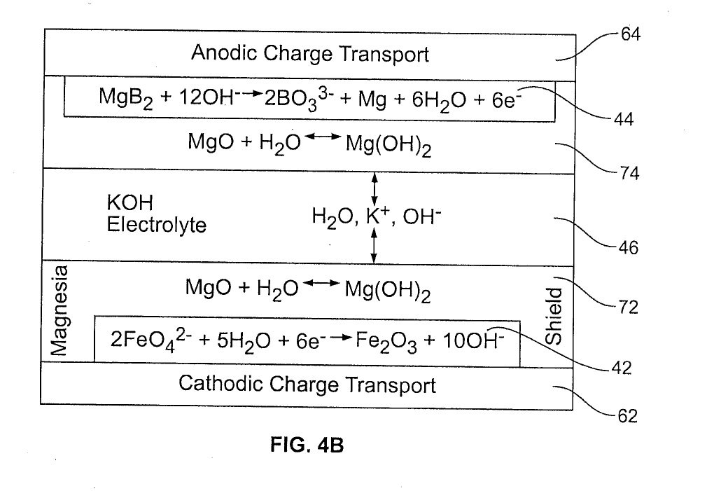

FIG. 4A depicts a prophetic example of a stent cross-section having an iron (VI) cathode 42 (e.g., K₂FeO₄ or K₃Na(FeO₄)₂) combined with KMnO₄, a KOH electrolyte 46, and a metal boride anode 34 (e.g., MgB₂). FIG. 4B is a schematic illustration of the redox electrochemistry involved in the electrochemical cell design of FIG. 4A. The insulating layer 36 and the encapsulating layer 56 depicted in FIG. 4A include PLA. Both the first and second bioerodible metal portions 32 and 34 comprise metallic iron. When activated, the iron (VI) cathode is placed in electrical contact with the first bioerodible metal portion 32 via cathode contact point 52 and the metal boride anode 34 is placed in electrical contact with the second bioerodible metal portion 34 via anode contact point 54. An iron cathode charge collector 62 is adjacent the cathode contact point 52 to facilitate electron transfer between the cathode 42 and the first bioerodible metal portion 32. An anode charge collector 64 is adjacent the anode contact point 54 to facilitate electron transfer between the anode 44 and the second bioerodible metal portion 34. The anode current collector 64 can also be made of iron. Magnesia shields 72 and 74 also separate the KOH electrolyte 46 from the cathode 42 and the anode 44 respectively.

The arrangement of the prophetic example of FIGS. 4A and 4B is designed to completely bioerode within a physiological environment into benign constituents that are absorbed into the body. As described above, the iron (VI) is reduced by the anode to become benign ferric oxide (Fe^{III}₂O₃) and the anode is oxidized to become borax, which is approved in some countries (excluding the US) as a food additive. The presence of the KOH improves the stability of the iron (VI) compound, especially if the cathode is free of nickel(II) and cobalt(II) impurities. The KMnO₄ mixed with the iron (VI) compound provides an additional charge transfer path to improve the efficiency of power transfer. The magnesia shield 74 prevents the metal boride anode 44 from uncontrollably reacting with the KOH electrolyte 46. Furthermore, the PLA insulating layer 36 and encapsulating layer 56 furthermore degrade into acidic degradation products that can neutralize the KOH electrolyte to buffer the electrochemical cell 40 as it completely degrades.

FIG. 5A depicts a prophetic example of a stent cross-section having nano-sized higher surface area lipscombite Fe²⁺Fe₂³⁺(PO₄)₂(OH)₂ iron phosphate cathode 42, a magnesium metal anode 44, and a polysaccharide polymer electrolyte 46 including magnesium chloride salt. FIG. 5B is a schematic illustration of the electrochemical intercalation involved in such a electrochemical cell. Nano-sized lipscombite iron phosphate can provide a tunnel structure that provides for a highly efficient cathode. Furthermore, the iron phosphate cathode, the polysaccharide electrolyte, and the magnesium anode can biodegrade into benign byproducts. The electrolyte 46 acts as a separator and also provides magnesium cations that migrate to the cathode from the magnesium anode 46. Because the anode is metallic magnesium, an additional charge collector is not needed for the anode, but a charge collector 62 is used for the cathode 42. The insulating layer 36 and the encapsulating layer 56 depicted in FIG. 5A include PLA. Both the first and second bioerodible metal portions 32 and 34 comprise metallic magnesium. Depending on compatibility with the cathode material, the cathode charge collector can be made of magnesium in some embodiments. The stent strut is also encapsulated with an outer layer 80 that includes a corrosion delaying material. An outer layer 80 can delay the erosion of the magnesium metal until the bioerodible body is at least partially endothelialized.

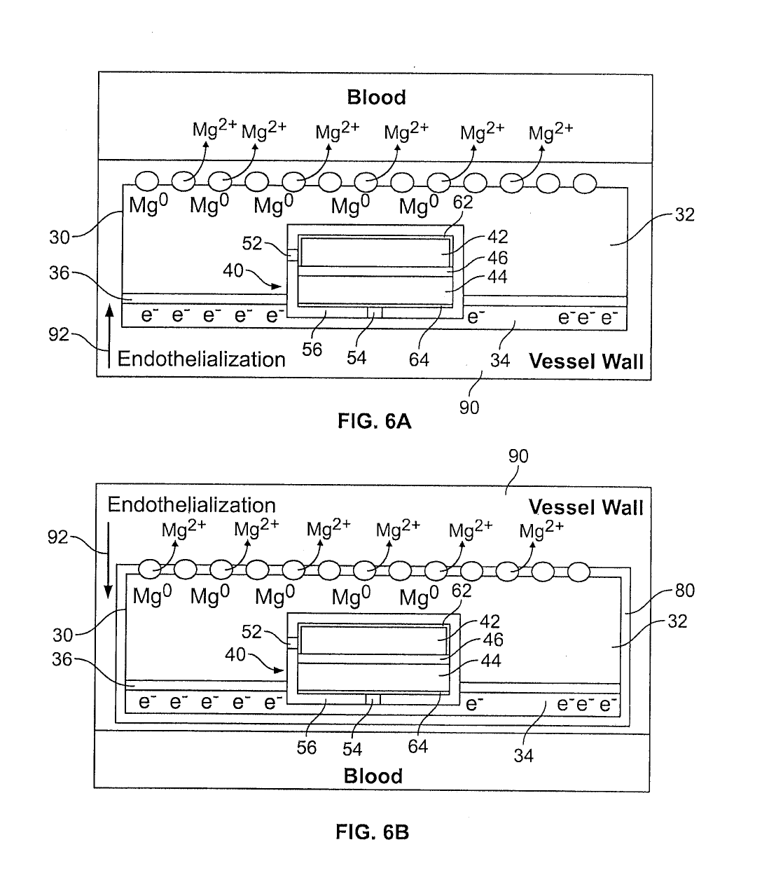

FIGS. 6A and 6B depict the stent can be implanted against a vessel wall 90. As shown in FIG. 5A, the first bioerodible metal region 32 can be positioned abluminally to contact the vessel wall 90, with the second bioerodible metal region 34 being positioned on a luminal side of the strut. In other embodiments, as shown in FIG. 5B, the first bioerodible metal region 32 can be positioned away from the vessel wall and the second bioerodible metal region 34 can be posited abluminally to contact the vessel wall 90. In both embodiments, endothelialization can occur (the cells of the vessel wall 90 can envelop the strut) as shown with arrow 92.

Initially, the stent 20 is implanted prior to activation of the electrochemical cells 40, as shown in FIG. 2. The electrochemical cell 40, however, can be activated after implantation. For example, the electrochemical cell 40 can be activated once endothelialization is completed (e.g., the stent 20 is completely enveloped by vessel wall cells). Prior to activation the electrochemical cell 40 does not provide a current to the bioerodible body 30, but the bioerodible body 30 and/or any outer coating can bioerode due to other biological and chemical processes.

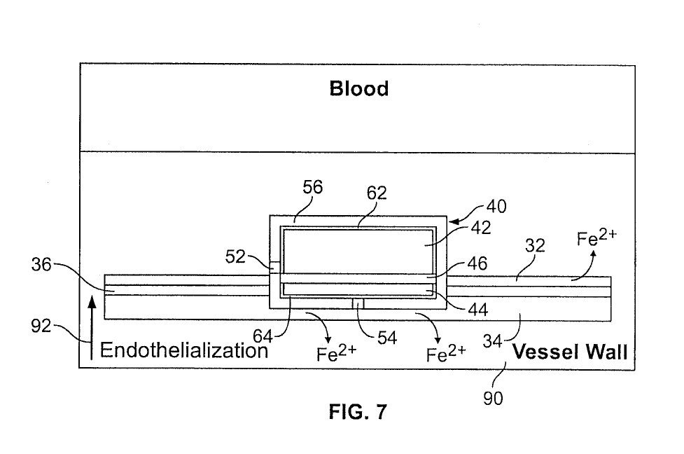

FIGS. 6A and 6B depict the electrochemical cells 40 in an activated condition and the struts fully endothelialized. Cathode contact point 52 and anode contact point 54 provide a low resistance path for electrons to flow between the electrodes and the portions of the bioerodible body. Due to the flow of electrons from the first bioerodible metal portion 32 to cathode 42, the bioerodible metal is converted into positively charged ions that dissolve in to the surrounding tissue and possibly into the blood stream. As shown, the bioerodible body 30 is formed of magnesium, but the bioerodible body can include iron or various other bioerodible metals and bioerodible alloys in other embodiments. This results in the first bioerodible metal portion 32 being anodically dissolved in an accelerated and controlled manner until the electrical connection between the cathode 42 and the first bioerodible metal portion 32 is disrupted due to the erosion of the first bioerodible metal portion 32, as shown in FIG. 7

FIG. 7 depicts an embodiment of a degraded strut after the activated electrochemical cell has lost electrical connection with a bioerodible metal portion. At this point, the bioerodible body ceases to erode due to anodic dissolution, but instead erodes due to other biological and chemical processes. The insulating layer 36 and encapsulating layer 56, as exposed, can degrade to form acidic by products, which can neutralize any alkaline materials (e.g., metal hydroxide electrolyte and metal bioerosion byproducts). Metal boride anodes can degrade into borax. Iron (VI) cathode can degrade into ferric oxide.

The composite stents can be made by many processes, including atomic layer deposition, laser deposition, and nanoparticle deposition.

Stent 20 can also be dyed or rendered radiopaque by addition of, e.g., radiopaque materials such as barium sulfate, platinum or gold, or by coating with a radiopaque material. Various radiopaque materials can be bioerodible under certain circumstances. For example, organic compounds can be substituted with iodine to render the compound radiopaque. In other embodiments, non-bioerodible radiopaque materials can be incorporated along with bioerodible materials. For example, magnetic Fe—Pt-nanoparticles can be used to impart radiopacity and also can allow for easy removal by use of a magnetic field.

Stent 20 can be configured for vascular, e.g., coronary and peripheral vasculature or non-vascular lumens. For example, they can be configured for use in the esophagus or the prostate. Other lumens include biliary lumens, hepatic lumens, pancreatic lumens, urethral lumens.

Stent 20 can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., see U.S. Pat. No. 6,290,721).

All publications, patent applications, patents, and other references mentioned herein are incorporated by reference herein in their entirety.

Still further embodiments are within the scope of the following claims.

What is claimed is:

1. A bioerodible endoprosthesis comprising:
a bioerodible body comprising a bioerodible metal; and
a bioerodible electrochemical cell comprising: a cathode and an anode, and an electrolyte between the cathode and the anode, the cathode being adapted to be in electrical contact with at least a first portion of the bioerodible body when the electrochemical cell is activated to accelerate the bioerosion of the first portion of the bioerodible body when the endoprosthesis is implanted within a physiological environment.

2. The endoprosthesis of claim 1, wherein the cathode comprises an iron(VI) compound.

3. The endoprosthesis of claim 2, wherein the cathode comprises an iron(VI) salt selected from the group consisting of K₂FeO₄, K₃Na(FeO₄)₂, BaFeO₄, and combinations thereof.

4. The endoprosthesis of claim 2, wherein the cathode further comprises KMnO₄.

5. The endoprosthesis of claim 2, wherein the cathode is free of nickel(II) and cobalt(II).

6. The endoprosthesis of claim 1, wherein the cathode comprises iron phosphate or an olivine metal phosphate.

7. The endoprosthesis of claim 1, wherein the electrolyte comprises KOH.

8. The endoprosthesis of claim 1, wherein the electrolyte comprises a polysaccharide polymer and a salt.

9. The endoprosthesis of claim 1, wherein the anode comprises a material selected from the group consisting of Mg, Zn, MgB₂, and FeB.

10. The endoprosthesis of claim 1, wherein the encapsulated electrochemical cell further comprises a shield disposed between the electrolyte and the cathode or between the electrolyte and the anode.

11. The endoprosthesis of claim 10, wherein the shield comprises magnesia or zirconia.

12. The endoprosthesis of claim 1, wherein the encapsulated electrochemical cell is bioerodible.

13. The endoprosthesis of claim 1, wherein the bioerodible body comprises a first bioerodible portion and a second bioerodible portion, wherein the electrochemical cell is embedded between the first bioerodible portion and the second bioerodible portion, wherein the cathode is adapted to be in electrical contact with the first bioerodible portion and the anode is adapted to be in electrical contact with the second bioerodible portion when the electrochemical cell is activated.

14. The endoprosthesis of claim 13, further comprising an insulating layer between the first bioerodible portion and the second bioerodible portion.

15. The endoprosthesis of claim 14, wherein the insulating layer comprises a polymer selected from the group consisting of polylactic acid, poly(lactic-co-glycolic acid), Poly(propylene-ram-ε-caprolactone carbonate), polycaprolactone, poly-L-lactic-acid, poly(3-hydroxybutyrate) and combinations thereof.

16. The endoprosthesis of claim 13, wherein the first bioerodible portion and the second bioerodible portion comprise different bioerodible metals.

17. The endoprosthesis of claim 13, wherein the first bioerodible portion and the second bioerodible portion comprise comprises the same bioerodible metal.

18. The endoprosthesis of claim 13, wherein the first bioerodible portion comprises metallic iron or a bioerodible iron alloy.

19. The endoprosthesis of claim 1, wherein the endoprosthesis is a stent comprising a plurality of struts, wherein at least one strut comprises at least a portion of the first bioerodible portion, at least a portion of the second bioerodible portion, and at least a portion of the encapsulated electrochemical cell.

20. The endoprosthesis of claim 1, wherein the cathode comprises an iron(VI) compound, the electrolyte comprises a metal hydroxide, and the anode comprises a metal boride.
